(19) United States
(12) Patent Application Publication
(10) Pub. No.: US 2004/0002095 A1
(43) Pub. Date: Jan. 1, 2004

(54) Universal signal amplification tail

(75) Inventors: Jason Liu, Concord, CA (US); John Wesley Breneman III, Brentwood, CA (US); Arlene Carillo, Vallejo, CA (US); Craig Hixson, Benecia, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 10/394,492

(22) Filed: Mar. 20, 2003

Related U.S. Application Data
(60) Provisional application No. 60/367,083, filed on Mar. 22, 2002.

(51) Int. Cl. C12Q 1/68; C07H 21/04; C12P 19/34; C07K 16/46

(52) U.S. Cl. 435/6; 435/91.2; 530/391.1; 536/23.1

(57) ABSTRACT

The present invention resides in a detection agent that can be used to amplify a detection signal for a target molecule. The detection agent contains a linear first single-stranded nucleic acid attached to a capture moiety that specific binds the target molecule of interest. The first single-stranded nucleic acid includes a plurality of repeat sequences, each of which is non-homopolymeric and can specifically bind to a second single-stranded nucleic acid that has at least one detectable label. Related method for using the claimed detection agent to amplify a detection signal and a kit for this purpose are also disclosed.

### CROSS-REFERENCES TO RELATED APPLICATIONS

This application claims the benefit of priority to U.S. Provisional Patent Application Serial No. 60/367,083, filed Mar. 22, 2002, the entire contents of which are hereby incorporated by reference.

### BACKGROUND OF THE INVENTION

A large variety of methods are known in the field of biomedical and genetic research to detect the presence of a macromolecule such as a nucleic acid or a protein in a sample. Hybridization between two single-stranded nucleic acids based on Watson-Crick base-pairing and immunoassays based on antigen-antibody interaction are two examples of these well known methods. When a molecule of interest is present in a nearly undetectable amount due to the sensitivity of the detection methods, amplification techniques become necessary. Currently available methods for amplification of a nucleic acid include polymerase chain reaction (PCR), ligase chain reaction (LCR), transcription-mediated amplification, self-sustained sequence replication or nucleic acid sequence-based amplification (NASBA), Rolling Circle Amplification (RCA), and the more recently developed branched-DNA technology.

While the widely employed gene amplification techniques are powerful tools in detecting a target nucleic acid in a very small quantity, they present some drawbacks. For instance, they often require expensive enzymes, e.g., thermostable DNA polymerases or ligases. Moreover, the use of these gene amplification techniques is limited to the detection of nucleic acids and not applicable in the detection of other macromolecules such as proteins. Thus, there exists a need to develop a universal signal amplification method that is more cost-effective and applicable to the detection of any macromolecule. The present invention addresses this and other needs.

### BRIEF SUMMARY OF THE INVENTION

The present invention resides in a novel approach for amplifying detection signals of a target molecule of interest. One aspect of the present invention is a detection agent, which includes two portions that are attached to each other. The first portion of the detection agent is a capture moiety that specifically binds to a target molecule. The second portion is a linear single-stranded nucleic acid that contains multiple repeat sequences. Each of the repeat sequences is a non-homopolymeric polynucleotide sequence and can specifically bind to a second single-stranded nucleic acid that has at least one detectable label. The two portions of the detection agent may be attached by a variety of means, such as by covalent bonds or non-covalent bonds.

In some embodiments, the capture moiety is a nucleic acid, a protein, or a carbohydrate. In a preferred embodiment, the capture moiety is an antibody. In some embodiments, each of the repeat sequences is about 5 to about 100 bases in length. In a preferred embodiment, each of the repeat sequences is about 50 bases in length. In some embodiments, the second single-stranded nucleic acid has multiple detectable labels. In some other embodiments, the repeat sequences are contiguous. In a preferred embodiment, the linear first single-stranded nucleic acid includes polynucleotide sequence of SEQ ID NO:1, each of the repeat sequences is SEQ ID NO:2, the target molecule is Thyroid Stimulating Hormone (TSH), the capture moiety includes and antibody against TSH, and the second single-stranded nucleic acid has three detectable labels.

A second aspect of the invention is a method for amplifying detection signals for a target molecule. The method includes the first step of contacting a detection agent with a sample, where the detection agent includes two portions attached to each other. The first portion of the detection agent is a capture moiety that specifically binds to a target molecule. The second portion is a linear single-stranded nucleic acid that contains multiple repeat sequences. Each of the repeat sequences is a non-homopolymeric polynucleotide sequence and can specifically bind to a second single-stranded nucleic acid that has at least one detectable label. The two portions of the detection agent may be attached by a variety of means, such as by covalent bonds or non-covalent bonds. The method also includes the second step of contacting the detection agent with the second single-stranded nucleic acid under suitable conditions such that the repeat sequences specifically binds to the second single-stranded nucleic acid.

In some embodiments, the capture moiety is a nucleic acid, a protein, or a carbohydrate. In a preferred embodiment, the capture moiety is an antibody. In some embodiments, each of the repeat sequences is about 5 to about 100 bases in length. In a preferred embodiment, each of the repeat sequences is about 50 bases in length. In some embodiments, the second single-stranded nucleic acid has multiple detectable labels. In some other embodiments, the repeat sequences are contiguous. In a preferred embodiment, the linear first single-stranded nucleic acid includes polynucleotide sequence of SEQ ID NO:1, each of the repeat sequences is SEQ ID NO:2, the target molecule is Thyroid Stimulating Hormone (TSH), the capture moiety includes and antibody against TSH, and the second single-stranded nucleic acid has three detectable labels.

A third aspect of the present invention is a kit for amplifying detection signals for a target molecule. The first component of the claimed kit is a detection agent. The first portion of the detection agent is a capture moiety that specifically binds to a target molecule. The second portion is a linear single-stranded nucleic acid that contains multiple repeat sequences. Each of the repeat sequences is a non-homopolymeric polynucleotide sequence and can specifically bind to a second single-stranded nucleic acid. The two portions of the detection agent may be attached by a variety of means, such as by covalent bonds or non-covalent bonds. The second component of the kit is the second single-stranded nucleic acid that has at least one detectable label.

In some embodiments, the capture moiety is a nucleic acid, a protein, or a carbohydrate. In a preferred embodiment, the capture moiety is an antibody. In some embodiments, each of the repeat sequences is about 5 to about 100 bases in length. In a preferred embodiment, each of the repeat sequences is about 50 bases in length. In some embodiments, the second single-stranded nucleic acid has multiple detectable labels. In some other embodiments, the repeat sequences are contiguous. In a preferred embodiment, the linear first single-stranded nucleic acid includes polynucleotide sequence of SEQ ID NO:1, each of the repeat sequences is SEQ ID NO:2, the target molecule is Thyroid Stimulating Hormone (TSH), the capture moiety includes and antibody against TSH, and the second single-stranded nucleic acid has three detectable labels.

Furthermore, the present invention provides a method for detecting genetic variations at predetermined loci in an individual. A DNA sample, e.g., a genomic DNA or cDNA sample, from a subject being tested is first obtained and the nucleic acid of the region containing the genetic variation is amplified by, e.g., polymerase chain reaction (PCR) or linked linear amplification (LLA). One version of the variation (complementary to X), if exists, is amplified so that a second fluorophore is attached (e.g., by using a fluorophore-labeled primer). Whereas the second version of the variation (complementary to Y), if exists, is amplified without any fluorescent groups attached. Upon hybridization of the detection agent comprising USAT and the sample following amplification, the amplified region, which may be an X or Y variation, binds to one of the two types of repeat sequences but not the other. Because of fluorescent resonance energy transfer (FRET), distinct fluorescent signals can be detected whether hybridization occurs at X, Y, or both types of repeat sequences. The individual's genetic variation in the region can thus be determined. The amplification of detection signal can be achieved by using a USAT comprising multiple copies of each type of the repeat sequences.

### BRIEF DESCRIPTION OF THE DRAWINGS

FIG. 1 is a schematic representation of USAT assembly using recombinant DNA technology and amplification of detection signal by hybridization between the USAT and multiple probes.

FIG. 2 describes the steps of constructing an exemplary USAT containing Alu sequence of the human genome as the repeat sequence.

FIG. 3 depicts the covalent attachment of a USAT with a modified base to a capture moiety, an antibody.

FIG. 4 depicts the non-covalent attachment of a USAT to a capture moiety, an antibody, via Watson-Crick base-pairing.

FIG. 5 shows the USAT sequence from human chromosome 1, the 47 bp repeat sequence, and the alignment of mini probes 1D, 2D, and 3B.

FIG. 6 shows the alignment of mini probes within the 47 bp repeat sequence and the locations of biotin moieties on the probes.

FIG. 7 depicts the relations between the detection signals and probe combinantions in an ELISA system.

### DEFINITIONS

The term “nucleic acid” or “polynucleotide” refers to deoxyribonucleotides or ribonucleotides and polymers thereof. The term also encompasses mimetics or analogs of nucleotides and their polymers, as well as polymers of nucleotides and nucleotide mimetics. Unless otherwise indicated, the term “nucleic acid” as used herein encompasses both single- and double-stranded forms. Anything that does not fall within the definition of “nucleic acid” or “polynucleotide” is regarded as “non-nucleic acid” or “non-polynucleotide” in this application.

The terms “protein,” “peptide,” and “polypeptide” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds. These terms further encompass polymers of amino acids, amino acid mimetics, and modified amino acids that also contain non-amino acid spacers and maintain the orientation of amino acids/analogs in the polymers.

The term “carbohydrate” as used herein refers to any compound having the general formula of C_{X}H_{2X}O_{X}. This term also encompasses derivatives of a carbohydrate, such as a carbohydrate with one or more substituent groups.

The term “linear” when used herein to describe a first single-stranded nucleic acid refers to its secondary structure. A linear single-stranded nucleic acid molecule contains no branch and has only one 5′ terminus and one 3′ terminus.

“A capture moiety that specifically binds to the target molecule” as used herein encompasses all molecules that bind to the target with sufficient specificity. The definition for specific binding is provided below. “A capture moiety that specifically binds to the target molecule” may be a protein, a nucleic acid, or a carbohydrate, and it is preferably a non-nucleic acid. For example, such a capture moiety may be an antibody for an antigen, which is the target molecule.

The term “repeat sequence” as used herein refers to a short polynucleotide sequence that appears multiple times in the first single-stranded nucleic acid of the claimed detection agent. These “repeat sequences” are generally between about 5 to about 100 bases and more preferably about 50 bases in length. Most preferably, the repeat sequence is SEQ ID NO:1. In some preferred embodiments, the repeat sequences are present in a USAT as a concatemer, i.e., X-X-X-X-X-X, where X is a discrete repeat sequence. There may be more than one kind of repeat sequence, i.e., one distinct polynucleotide sequence, within the first single-stranded nucleic acid. For example, two different repeat sequences X and Y may be present in a USAT in the form of X-Y-X-Y or X-X-Y-Y. Unless otherwise indicated, the different repeat sequences may be present in any order in a USAT. The “repeat sequences” may be linked directly to one another without any spacing, or may be “contiguous”; they may also be “noncontiguous,” i.e., linked via various linkers, such as one or more nucleotides and non-polynucleotides. Furthermore, one or more repeat sequences in a USAT may each contain a detectable label, e.g., a fluorescent moiety, in some embodiments of the present invention.

The term “specifically bind” or “specific binding” when used in the context of describing the binding relationship between two binding partners, e.g., a target molecule and an element that specifically binds it, or the repeat sequences of a first single-stranded nucleic acid and a second single-stranded nucleic acid, refers to a binding reaction to one binding partner that is determinative of the presence of the other binding partner in the presence of a heterogeneous population of other molecules. Thus, under designed assay conditions, a first binding partner binds to a second binding partner but does not bind in a significant amount to other molecules present in the sample. Specific binding may be ensured by prior selection of binding partners. For example, specific binding to an antibody under proper assay conditions may require an antibody that is selected for its specificity for a particular protein. Both monoclonal and polyclonal antibodies may be used for specific binding in practicing the present invention. Antibodies raised against a polypeptide of interest can be selected to obtain antibodies specifically immunoreactive with that polypeptide and not with any other polypeptides. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane, Antibodies, A Laboratory Manual, Cold Spring Harbor Publications, NY, 1988, for a description of immunoassay formats and conditions that can be used to determine specific immunoreactivity. Typically, a specific or selective reaction will be at least twice the background signal or noise and more typically more than 10 to 100 times background.

The term “antibody” denotes a protein of the immunoglobulin family or a polypeptide including fragments of an immunoglobulin that is capable of noncovalently, reversibly, and in a specific manner binding a corresponding antigen. An illustrative antibody structural unit includes a tetramer. Each tetramer is composed of two identical pairs of polypeptide chains, each pair having one “light” (about 25 kD) and one “heavy” chain (about 50-70 kD), connected through one or more disulfide bonds. The recognized immunoglobulin genes include the κ, λ, α, γ, δ, ε, and μ constant region genes, as well as the myriad immunoglobulin variable region genes. Light chains are classified as either κ or λ Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively.

The term “complementarity-determining domains” or “CDRs” refers to the hypervariable regions of V_{L} and V_{H}. The CDR is the immunogen-binding site of the antibody chain that harbors specificity for that immunogen, e.g., a protein, a carbohydrate, or even a nucleic acid (such as a Z DNA). There are three CDRs (CDR1-3, numbered sequentially from the N-terminus) in each human V_{L} or V_{H}, constituting about 15-20% of the variable domains. The CDRs are structurally complementary to the epitope of the immunogen and are thus directly responsible for the binding specificity. The remaining stretches of the V_{L} or V_{H}, the so-called framework regions, exhibit less variation in amino acid sequence (Kuby, Immunology, 4th ed., Chapter 4, W. H. Freeman & Co., New York, 2000).

The positions of the CDRs and framework regions are determined using various well known definitions in the art, e.g., Kabat, Chothia, International ImMunoGeneTics database (IMGT), and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al, Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

The terms “antibody light chain” and “antibody heavy chain” denote the V_{L} or V_{H}, respectively. The V_{L} is encoded by the gene segments V (variable) and J (junctional), and the V_{H} is encoded by V, D (diversity), and J. Each of V_{L} or V_{H} includes the CDRs as well as the framework regions.

Antibodies exist as intact immunoglobulins or as a number of well-characterized fragments produced by digestion with various peptidases. Thus, for example, pepsin digests an antibody below the disulfide linkages in the hinge region to produce F(_{ab})′₂, a dimer of F_{ab}′ which itself is a light chain joined to V_{H}-C_{H}1 by a disulfide bond. The F(_{ab})′₂ may be reduced under mild conditions to break the disulfide linkage in the hinge region, thereby converting the F(_{ab})′₂ dimer into an F_{ab}′ monomer. The F_{ab}′ monomer is essentially F_{ab} with part of the hinge region (Paul, Fundamental Immunology 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fᵥ) or those identified using phage display libraries (see, e.g., McCafferty et al., Nature, 348:552-554, 1990)

For preparation of monoclonal or polyclonal antibodies, any technique known in the art can be used (see, e.g., Kohler & Milstein, Nature, 256:495-497, 1975; Kozbor et al., Immunology Today, 4:72, 1983; Cole et al., Monoclonal Antibodies and Cancer Therapy, pp. 77-96. Alan R. Liss, Inc. 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies, and heteromeric F_{ab} fragments, or scFv fragments that specifically bind to selected antigens (see, e.g., McCafferty et al., supra; Marks et al., Biotechnology, 10:779-783, 1992).

The term “non-homopolymeric” when used to describe a repeat sequence within a linear first single-stranded nucleic acid of the claimed detection agent refers to the fact that the repeat sequence does not contain any recurring segment of more than three bases, preferably more than four bases, and more preferably more than five bases (such as ATGCGAT-ATGCGAT-ATGCGAT), particularly any stretch of more than five, preferably more than six, more preferably more than seven, and most preferably more than ten, of the same nucleotide or its mimetic (such as poly A or poly dT) in a continuous fashion. In other words, there exist no repeat segments of more than three bases within any one of the repeat sequences.

The term “sample” as used herein may include a biological sample as well as a sample of other origins. A biological sample refers to sections of tissues of a living organism such as biopsy and autopsy samples, and frozen sections taken for histological purposes. Such samples may include whole blood, serum, plasma, cerebrospinal fluid, sputum, tissue, cultured cells, e.g., primary cultures, explants, transformed cells, stool, urine, vesicle fluid, mucus, and other bodily secretion or tissue that could be sampled with a swab device. The term “sample” also encompasses any sample that may be the subject of testing in a biochemical or genetic assay, including a sample from a microorganism culture or a sample from the environment, e.g., from any body of water, air, soil, etc.

The “detectable label” as a part of the second single-stranded nucleic acid is a moiety that may directly or indirectly impart a detectable signal. A label may be detectable by spectroscopic, photochemical, biochemical, immunochemical, electrical, magnetic, optical, or chemical means. Directly detectable labels include fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads. Magnetic or ferrous particles/beads may also be used as direct labels and detected with a magnetometer. Indirectly detectable labels work via its specific binding partner with a direct detectable label. For example, a biotin moiety acts as an indirect label when a detectable signal results from a streptavidin-enzyme conjugate.

The term “contiguous” when used to describe the repeat sequences within the first single-stranded nucleic acid of the claimed detection agent refers to the fact that there is no space between any repeat sequence and its neighboring repeat sequence(s), i.e., the last base of a repeat sequence is directly linked to the first base of the next repeat sequence and so forth until the last repeat sequence. On the other hand, if the repeat sequences are not contiguous, that means there exists space between at least some of the repeat sequences. Such space may include one or more nucleotides or nucleotide analogs/mimetics, non-nucleotides such as one or more amino acids, polypeptides, carbohydrates, or other spacing moieties/constructs.

### DETAILED DESCRIPTION OF THE INVENTION

I. Introduction

The present application discloses a universal approach in the amplification of a detection signal. Briefly, the present invention provides a novel detection agent comprising a capture moiety conjugated to a linear single-stranded nucleic acid, which contains multiple non-homopolymeric repeat sequences. This single-stranded nucleic acid is referred to herein as a Universal Signal Amplification Tail, or USAT. Attached to the USAT is a capture moiety that can specifically bind to a target molecule of interest. This capture moiety, a specific binding partner of the target molecule under designated assay conditions, may have diverse structural and functional characteristics. For instance, this moiety may be a nucleic acid that hybridizes specifically with its complementary sequence (the target), a protein, e.g., an antibody that specifically recognizes an antigen (the target), or a carbohydrate that specifically binds another molecule (the target) in, e.g., a sugar that specifically binds to a lectin. This capture moiety may also be any other material, natural or synthetic, as long as it can specifically recognize a predetermined target of interest.

The USAT, which may be a naturally occurring sequence or an artificial sequence, includes multiple non-homopolymeric repeat sequences of polynucleotides. The non-homopolymeric repeat sequences, which are characterized as containing no recurring segments of more than three bases, provide distinct binding sites for multiple molecules of a polynucleotide probe, e.g., a nucleic acid of complementary sequence having at least one detectable label. Since each target molecule of interest specifically binds to a molecule of the capture moiety-USAT conjugate and each USAT in turn specifically binds to multiple probes via multiple binding sites offered by the repeat sequences, the signal for the target of interest is thus amplified for easier and more accurate detection.

Similar strategies for amplifying detection signals have been described in, e.g., U.S. Pat. Nos. 5,124,246 and 6,245,513. The present invention, however, provides some distinct advantages over these approaches. For instance, the signal amplification methods disclosed in U.S. Pat. No. 5,124, 246 are only applicable in nucleic acid hybridization assays, not in other assay systems, such as an immunoassay system. While the methods provided by U.S. Pat. No. 6,245,513 are applicable to amplifying a detection signal originated from a non-nucleic acid capture moiety, their reliance on homopolymeric repeat sequences, e.g., a polyA or a polydT sequence, for signal amplification and thus presents a major shortcoming: the lack of distinct binding sites in a homopolymeric sequence leads to insufficient specificity and efficiency in probe hybridization, and consequently, insufficient and unpredictable signal amplification. In contrast, the present application reveals, for the first time, that when non-homopolymeric repeat sequences are used to offer multiple distinct binding sites for multiple probes, much higher probe binding specificity and efficiency, and therefore, better quality signal amplification can be achieved.

Another aspect of the present invention is the use of USAT for detecting a genetic variation, such as a single nucleotide polymorphism (SNP), with the option of amplifying the detection signal. In this aspect, the first single-stranded nucleic acid, or USAT, contains more than one type of repeat sequence. For instance, a USAT may include two types of repeat sequences, which are of the same or similar length but have at least one different nucleotide. Each type of repeat sequence corresponds to a known variation of a gene of interest within a particular region of, e.g., about 50 nucleotides. One end of the USAT is attached to a capture moiety that allows the easy isolation or identification of the detection agent, e.g., a bi-colored bead containing a predetermined ratio of two dyes, via a covalent bond or a non-covalent bond (such as the binding between two single-stranded nucleic acids through Watson-Crick base-pairing). Each of one type of the repeat sequences (for example, X) is labeled with a detectable group, e.g., a first fluorophore, but not the other type of repeat sequences (e.g., Y).

On the other hand, a sample, e.g., a genomic DNA or cDNA sample, from a subject whose genetic variation is to be tested is first obtained and the nucleic acid of the region containing the genetic variation is amplified by, e.g., polymerase chain reaction (PCR) or linked linear amplification (LLA). One version of the variation (complementary to X), if exists, is amplified so that a second fluorophore is attached (e.g., by using a fluorophore-labeled primer). Whereas the second version of the variation (complementary to Y), if exists, is amplified without any fluorescent groups attached. Upon hybridization of the detection agent comprising USAT and the sample following amplification, the amplified region, which may be an X or Y variation, binds to one of the two types of repeat sequences but not the other. Because of fluorescent resonance energy transfer (FRET), distinct fluorescent signals can be detected whether hybridization occurs at X, Y, or both types of repeat sequences. The individual's genetic variation in the region can thus be determined. The amplification of detection signal can be achieved by using a USAT comprising multiple copies of each type of the repeat sequences.

All references cited in this application are hereby incorporated by reference in their entirety.

II. Construction of the Linear First Single-Stranded Nucleic Acid

A. Naturally Occurring Sequences

The first component of the detection agent of the present application is a linear single-stranded nucleic acid. Certain naturally occurring sequences may be used as this so-called “the first single-stranded nucleic acid.” For example, it is well known that human genome contains a large number of repetitive sequences, such as SEQ ID NO:2 found in chromosome 1. A variety of cloning methods, including polymerase chain reaction (PCR)-based cloning methods, may be used to obtain such repetitive sequences from, e.g., a human genomic DNA library. Methods for making and screening genomic and cDNA libraries are described in numerous publications (see, e.g., Benton and Davis, Science 196:180-182, 1977; Gubler and Hoffman, Gene 25:263-269, 1983).

Using the methods well known to those of skill in the art, a cloned genomic sequence can further be subcloned into an appropriate vector, such as a plasmid, for amplification of the sequence. The production of a single-stranded nucleic acid suitable for practicing the present invention can be achieved by, e.g., synthesizing the single-stranded nucleic acid using a single-stranded phage vector (such as M13) or by separating the strands of a double-stranded nucleic acid on a denaturing gel. A single-stranded DNA can be obtained using asymmetric (i.e., one directional) PCR. A single-stranded RNA, e.g., generated in an in vitro transcription system, may also be suitable for practicing the present invention.

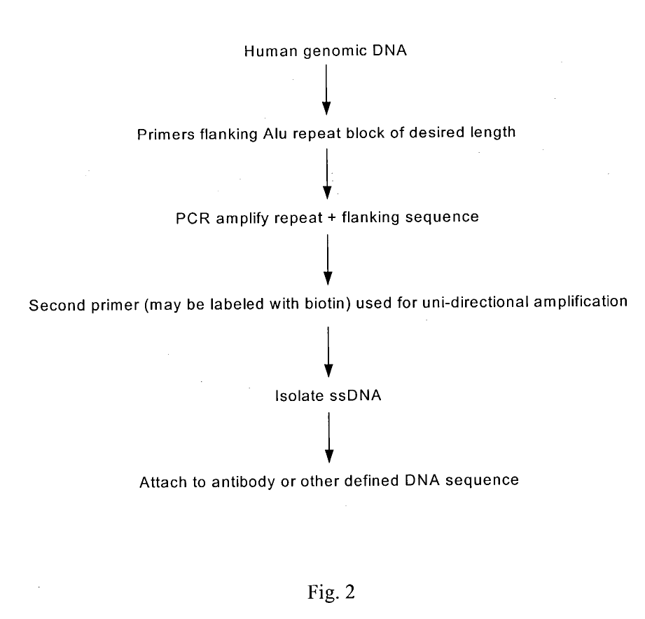

In addition, well established mutagenesis techniques (such as site-directed mutagenesis) can be used to modify any naturally occurring polynucleotide sequences to better serve the purpose in accordance with the present invention. For instance, the non-conserved Ts as shown in FIG. 2 can be replaced by Cs to restore full conservancy and better signal amplification.

For a general description of recombinant technology including cloning of a genomic sequence, subcloning, and production of a single-stranded nucleic acid, see, e.g., Sambrook and Russell, Molecular Cloning: A Laboratory Manual 3d ed. 2001; Kriegler, Gene Transfer and Expression: A Laboratory Manual 1990; and Ausubel et al., Current Protocols in Molecular Biology 1994.

B. Artificial or Synthetic Sequences

It will be appreciated that the first single-stranded nucleic acid of the present invention may also be an artificial origin. One option is synthesize the first single-stranded nucleic acid in its entirety. For instance, polynucleotides of a desired sequence may be chemically synthesized according to the solid phase phosphoramidite trimester method first described by Beaucage and Caruthers, Tetrahedron Lett. 22:1859-1862, 1981, using an automated synthesizer, as described by Van Devanter et al., Nucleic Acids Res. 12:6159-6168, 1984. Purification of polynucleotides can be achieved by either native acrylamide gel electrophoresis or by anion-exchange HPLC as described in Pearson and Teanier, J. Chrom. 255:137-149, 1983.

The other option is to synthesize the pieces of the first single-stranded nucleic acid (such as the repeat sequences) separately and then connect the pieces through a ligation process (such as by a chemical or an enzymatic process, e.g., using T4 ligase or RNA ligase). Repeat sequences of different polynucleotide sequences may be present in the same first single-stranded nucleic acid. For example, two different types of repeat sequences may exist in the form of X-Y-X-Y-X-Y or X-X-X-Y-Y-Y in the first single-stranded nucleic acid. The general methods for chemical synthesis and purification are the same as described above. This synthesis-ligation approach may provide certain advantages, such as the flexibility in the number of repeat sequences and whether the repeat sequences are contiguous. If the repeat sequences are non-contiguous, they may be connected via one or more nucleotides/nucleotide analogs, or a non-polynucleotide such as one or more amino acids/polypeptides. Other linkers such as carbon linkers may be also be used for this purpose.

III. The Capture Moiety That Specifically Binds to the Target Molecule

The second component of the detection agent of the present invention is a capture moiety that can specifically bind to a target molecule, i.e., the subject of detection. This capture moiety, attached to the first single-stranded nucleic acid, may be one of the two partners of any specific interaction under designated conditions, whereas the target molecule is the other partner. Examples of such specific interaction include antigen-antibody, a nucleic acid and its complementary sequence, and ligand-receptor interactions. Thus, depending on what the intended detection target is, a capture moiety maybe a nucleic acid, or a non-nucleic acid such as a polypeptide or a carbohydrate. A capture moiety may even be a suitable synthetic material with sufficient specificity to the target molecule.

A. A Polynucleotide as the Capture Moiety

If the desired capture moiety is a polynucleotide, it may be obtained recombinantly following directly cloning or synthetically by chemical methods. Both methods are described in an earlier section.

B. A Non-Polynucleotide as the Capture Moiety

If the desired capture moiety is a non-polynucleotide, e.g., a protein (such as an antibody) or a carbohydrate (such as a carbohydrate that specifically binds a particular lectin), it may be obtained through isolation and purification from a natural source, or through an artificial means, such as recombinant production of a protein or chemical synthesis of a carbohydrate.

1. Purification of a Capture Moiety from a Natural Source

Depending on the nature of the intended capture moiety, various well known methods may be suitable for isolating and purifying the molecule from a natural source. For instance, when such capture moiety is a protein, the standard protein purification methods, as outlined below, can be used. Because of its known specific binding partner, a non-protein capture moiety, such as a carbohydrate, may also be easily isolated and purified in any affinity-based purification system such as affinity chromatography regardless of its nature.

Solubility Fractionation

Salt fractionation can be used as an initial step to separate a desired protein from other unwanted proteins. The preferred salt is ammonium sulfate, which precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The desired protein is precipitated at an appropriate ammonium sulfate concentration according to its hydrophobicity and is then solubilized in a buffer with the excess salt removed if necessary, through either dialysis or diafiltration. Other methods that rely on solubility of proteins, such as cold ethanol precipitation, are well known to those of skill in the art and may also be used to prepare a protein fraction from a mixture of a large number of proteins, such as a cell lysate.

Size Differential Filtration

Based on a predicted molecular weight, a protein can be isolated from other proteins of greater and lesser sizes using ultrafiltration through membranes of different pore sizes (for example, Amicon or Millipore membranes). As a first step, a protein mixture (e.g., a serum or a cell lysate) is ultrafiltered through a membrane with a pore size that has a lower molecular weight cut-off than the predicted molecular weight of the desired protein. The retentate of the ultrafiltration is then ultrafiltered against a membrane with a molecular cut-off greater than the predicted molecular weight of the desired protein. The protein will pass through the membrane into the filtrate, which can then be processed in a next step of column chromatography.

Column Chromatography

A protein used for as a capture moiety in constructing the claimed detection agent can also be separated from other proteins on the basis of their size, net surface charge, hydrophobicity, and affinity for ligands. Column chromatography is a frequently used method. For example, antibodies can be isolated from other non-antibody proteins using a column with immobilized protein A or protein G, which are bacterial cell wall proteins that bind to a domain in the Fc region of antibodies. Furthermore, antibodies against different antigens can be separated based on their distinct affinity to these antigens, which are immobilized to a column in a preferred format of column chromatography for antibody purification. All of these methods are well known in the art, and it will be apparent to one of skill that chromatographic techniques can be performed at any scale and using equipment from many different manufacturers (e.g., Pharmacia Biotech).

Production of Antibodies as Capture Moieties

In some preferred embodiments of the present invention, the capture moiety is an antibody specifically reactive to a target molecule of interest, e.g., the human thyroid stimulating hormone (TSH). Methods for producing polyclonal and monoclonal antibodies that react specifically with an immunogen of interest are known to those of skill in the art (see, e.g., Coligan, Current Protocols in Immunology Wiley/Greene, NY, 1991; Harlow and Lane, Antibodies: A Laboratory Manual Cold Spring Harbor Press, NY, 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256:495-497, 1975). Such techniques include antibody preparation by selection of antibodies from libraries of recombinant antibodies in phage or similar vectors (see, Huse et al., Science 246:1275-1281, 1989; and Ward et al., Nature 341:544-546, 1989). Another means of purifying a desired antibody may be achieved by labeling the target molecule, e.g., a protein or a nucleic acid, with streptavidin. Purified antibody can then be obtained using the iminobiotin-streptavidin purification system as marketed by Pierce Biotechnology.

In order to produce antisera containing antibodies with desired specificity for the construction of a detection agent of this invention, the antigen of interest (e.g., TSH) or an antigenic fragment thereof can be used to immunize suitable animals, e.g., mice, rabbits, or primates. A standard adjuvant, such as Freund's adjuvant, can be used in accordance with a standard immunization protocol. Alternatively, a synthetic peptide derived from that particular antigen can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. When appropriate, blood with high titers of desired antibodies may also be collected from a human subject. Further fractionation of the antisera to enrich antibodies specifically reactive to the antigen and purification of the antibodies can be accomplished subsequently, see, Harlow and Lane, supra, and general descriptions of antibody purification offered below.

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al., supra. A more detailed description of antibody production by recombinant methods can be found in a later section.

2. Synthesis of a Capture Moiety

Various chemical methods are known in the art for synthesizing a non-nucleic acid capture moiety, such as a polypeptide or a carbohydrate. For example, peptides may be synthesized by solid-phase peptide synthesis methods using procedures similar to those described by Merrifield et al., J. Am. Chem. Soc., 85:2149-2156, 1963; Barany and Merrifield, Solid-Phase Peptide Synthesis, in The Peptides: Analysis, Synthesis, Biology Gross and Meienhofer (eds.), Academic Press, N.Y., vol. 2, pp. 3-284, 1980; and Stewart et al., Solid Phase Peptide Synthesis 2nd ed., Pierce Chem. Co., Rockford, Ill. 1984. During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Depending on the specific lectin as the target molecule of interest, various carbohydrates may be used as the capture moiety of the claimed detection agent. These carbohydrates may be obtained by chemical synthesis using methods known in the art, see, e.g., Collins and Ferrier, Monosaccharides: Their Chemistry and Their Roles in Natural Products, John Wiley & Sons, 1995; Khan et al. (Ed.) Modern Methods in Carbohydrate Synthesis, Gordon & Breach Publishing Group, 1996.

Additionally, a polypeptide may also be recombinantly produced and purified for constructing the detection agent of the present invention. The general methods for recombinantly producing antibodies with desired specificity are known to those skilled in the relevant art and are described in numerous publications. See, e.g., U.S. Pat. No. 5,665,570. Briefly, the genes encoding an antibody with desired specificity can be identified by screening a B cell cDNA library using various cloning techniques, e.g., a cloning method based on polymerase chain reaction (PCR), and subsequently expressed in suitable host cells. For a general description of recombinant DNA technology, see, e.g., Sambrook and Russell, Supra;; Kriegler, Supra; and Ausubel et al., Supra.

Another means for recombinantly producing antibodies with desired specificity relies on the chimeric antibody technology. Generally, the genes encoding the variable regions of a non-human monoclonal antibody (e.g., a murine antibody) are cloned and joined with the coding sequences for human constant regions to produce the so-called “humanized” antibodies. See, e.g., U.S. Pat. Nos. 5,502,167; 5,607,847; 5,773,247. Such humanized chimeric antibodies produced by host cells are suitable for constructing the claimed liquid IgG and IgM calibrators.

In addition, fully human antibodies against a specific antigen can be prepared by immunizing a transgenic animal that has been genetically manipulated so that its endogenous Ig loci has been inactivated and replaced with human Ig loci, to produce the antibodies in response to the antigenic challenge. Human monoclonal antibodies so produced are also suitable for practicing the present invention. Detailed description of this recently developed technology for producing human monoclonal antibodies of any desired specificity can be found in, e.g., U.S. Pat. Nos. 6,114,598; 6,150,584; 6,162,963. This approach differs from the first two in that it does not require expression of genes encoding an antibody with desired specificity in host cells; rather, fully human monoclonal antibodies can be obtained following the immunization procedure and antibody purification method outlined in the last section once a transgenic animal is established.

Host Cells

Various cell types, both prokaryotic and eukaryotic, are suitable for the expression of a recombinant antibody. These cell types include but are not limited to, for example, a variety of bacteria such as E. coli, Bacillus, and Salmonella, as well as eukaryotic cells such as yeast, insect cells, and mammalian cells. Suitable cells for gene expression are well known to those of skill in the art and are described in numerous scientific publications such as Sambrook and Russell, supra.

Expression Vectors

Upon acquisition of the nucleic acid sequences encoding a desired antibody, the sequences are typically cloned into an intermediate vector before transformation into prokaryotic or eukaryotic cells for replication and/or expression. The intermediate vector is typically a prokaryote vector such as a plasmid or shuttle vector.

To obtain high level expression of a cloned gene, such as the cDNA encoding an antibody with a desired specificity, one typically subclones the cDNA into an expression vector that contains a strong promoter to direct transcription, a transcription/translation terminator, and a ribosome binding site for translational initiation. Suitable bacterial promoters are well known in the art and fully described in scientific literature such as Sambrook and Russell, supra, and Ausubel et al, supra. Bacterial expression systems for expressing antibody chains of the recombinant catalytic polypeptide are available in, e.g., E. coli, Bacillus, and Salmonella (Palva et al., Gene, 22:229-235, 1983; Mosbach et al., Nature, 302:543-545, 1983). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the proteolytic antibody chain in host cells. A typical expression cassette thus contains a promoter operably linked to the nucleic acid sequence encoding the proteolytic antibody chain and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc or histidine tags.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a proteolytic antibody chain under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Transfection Methods

Standard transfection methods are used to produce bacterial, mammalian, yeast, or insect cell lines that express large quantity of the desired recombinant antibody, which is then purified using standard techniques (see, e.g., Colley et al., J. Biol. Chem., 264:17619-17622, 1989; Guide to Protein Purification, in Methods in Enzymology, vol. 182 (Deutscher, ed.), 1990) and as described above. Transformation of eukaryotic and prokaryotic cells are performed according to standard techniques (see, e.g., Morrison, J. Bact., 132:349-351, 1977; Clark-Curtiss and Curtiss, Methods in Enzymology, 101:347-362 (Wu et al., eds), 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least both genes into the host cell capable of expressing the recombinant catalytic polypeptide.

After the expression vector is introduced into the cells, the transfected cells are cultured under conditions favoring expression of the antibody with desired specificity, which is screened for (e.g. using hybridization assays and electrophoresis) and recovered from the culture using standard techniques identified below.

Purification of Recombinant Antibodies

The recombinant antibodies may be purified to substantial purity by standard techniques as described above, including selective precipitation with such substances as ammonium sulfate; column chromatography, gel filtration, immunopurification methods, and others (see, e.g., U.S. Pat. No. 4,673,641; Scopes, Protein Purification: Principles and Practice, 1982; Sambrook and Russell, supra; and Ausubel et al., supra).

IV. Attachment of the First Single-Stranded Nucleic Acid to the Capture Moiety

Upon acquiring the first single-stranded nucleic acid and the capture moiety that specifically binds to the target molecule, the two parts can be attached to produce the detection agent of this invention. Such attachment can be either direct or indirect, i.e., one or more linkers may be used to effectuate the connection.

Depending on the nature of the capture moiety, the presence of a linker, and possible modifications made to the first single-stranded nucleic acid, various covalent (such as a phosphodiester bond or a disulfide bond) or non-covalent bonds (such as an ionic bond, van der Waals force, an electrostatic bond, or a hydrogen bond) can be established to directly connect the two parts of the detection agent of the present invention. For instance, a polynucleotide capture moiety can be connected to a first single-stranded nucleic acid via a covalent bond, e.g., a phosphodiester bond. A non-nucleic acid capture moiety, e.g., a polypeptide, frequently has some functional groups, such as amine (—NH₂), carboxylic acid (—COOH), and sulfhydryl (—SH) groups, with which the functional groups of a linker or a modified group of a first single-stranded nucleic acid may easily react and establish a covalent bond that conjugates the capture moiety and the nucleic acid. To facilitate the conjugation process, suitable linkers known in the art may be used to provide necessary functional groups. A linker may be, for example, a straight or branched amino acid polymer, a straight- or branched-chain carbon linker, a heterocyclic carbon linker, or a polyether linker. Furthermore, the first single-stranded nucleic acid may be derivatized, e.g., via its hydroxyl groups, prior to conjugation to attach reactive functional groups, using any of a number of molecules such as those available from Pierce Chemical Company, Rockford, Ill.

Alternatively, a single-stranded nucleic acid may be linked to a capture moiety non-covalently via the known interaction of two binding partners: a tag and a tag-binder. One of the partners of this binding interaction, e.g., a tag, can be attached to the nucleic acid whereas the other partner, e.g., a tag binder, can be attached to the capture moiety. A number of tags and tag binders can be used, based upon known molecular interactions well described in the literature, so long as this binding does not interfere with the other binding interactions during the detection of the target molecule. For example, receptor-ligand interactions are appropriate as tag and tag-binder pairs. Agonists and antagonists of cell membrane receptors (e.g., cell receptor-ligand interactions such as transferrin, c-kit, viral receptor ligands, cytokine receptors, chemokine receptors, interleukin receptors, the cadherein family, the integrin family, the selectin family, and the like; see, e.g., Pigott & Power, The Adhesion Molecule Facts Book I, 1993) are also suitable binding pairs. Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors. In addition, some synthetic polymers, such as polyurethanes, polyesters, polycarbonates, polyureas, polyamides, polyethyleneimines, polyarylene sulfides, polysiloxanes, polyimides, and polyacetates can form an appropriate tag or tag binder as well.

V. Construction of the Labeled Second Single-Stranded Nucleic Acid

A. Acquisition of the Nucleic Acid

The same general methods for acquiring the first single-stranded nucleic acid as provided above are applicable to obtain a nucleic acid as the second single-stranded nucleic acid that has at least one detectable label. The length of the second single-stranded nucleic acid is the same as or shorter than that of the repeat sequence it is to hybridize with specificity under designated assay conditions.

B. Detectable Labels

Various binding assays such as nucleic acid hybridization assays or immunoassays often utilize a labeling agent to specifically bind to and label the binding complex formed by, e.g., two strands of complementary polynucleotide sequences or an antibody and its specific antigen. The labeling agent may itself be one of the moieties comprising the primary binding complex, such as an antigen/antibody complex, or may be a third moiety, such as another antibody, that specifically binds to the primary binding complex. A label may be detectable by spectroscopic, photochemical, biochemical, immunochemical, electrical, optical, or chemical means. Some examples are, but not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and calorimetric labels such as colloidal gold or colored glass or plastic (e.g., polystyrene, polypropylene, latex, etc.) beads.

Furthermore, since the second single-stranded nucleic acid forms, with the first single-stranded nucleic acid, a double-stranded structure when bound to the USAT sequence, it is possible to use a dye specific for double-stranded nucleic acids, i.e., a dye that fluoresces only when bound to a double-stranded nucleic acid, to indicate such hybridization.

In some immunoassays, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents in an immunoassay. These proteins are normal constituents of the cell walls of streptococcal bacteria. They exhibit a strong non-immunogenic reactivity with immunoglobulin constant regions from a variety of species (see, generally, Kronval, et al., J. Immunol., 111:1401-1406, 1973; and Akerstrom, et al., J. Immunol., 135:2589-2542, 1985).

C. Attachment to a Solid Support

In some embodiments, the labeled second single-stranded nucleic acid is immobilized to a solid support. A solid support is often a synthetic inert polymeric material, but may also be naturally-occurring. Examples of carrier material are acrylamide, cellulose, nitrocellulose, glass, polystyrene, polyethylene vinyl acetate, polypropylene, polymethacrylate, polyethylene, polysilicates, polyethylene oxide, polycarbonates, teflon, fluorocarbons, nylon, silicon rubber, collagen, polyanhydrides, polyglycolic acid, polylactic acid, polyorthoesters, polypropylfumarate, glycosaminoglycans, and polyamino acids. A solid support may be in one of the many useful forms including thin films or membranes, beads, bottles, dishes, fibers, woven fibers, shaped polymers, particles, and microparticles such as microspheres. Preferred forms of support are plates and beads. The most preferred form of beads is magnetic beads or latex beads.

The labeled second single-stranded nucleic acid can be attached to a solid support via various linkers. A linker can be attached to the 5′- or 3′-terminus of a nucleic acid. A linker can also be attached to a nucleic acid via an internal nucleotide. In the case of indirect linkage between a nucleic acid and a carrier, a linker may include a peptide or a branched amino acid polymer. There are other suitable linkers well known to those of skill in the art, including but not limited to, straight or branched-chain carbon linkers, heterocyclic carbon linkers, or polyether linkers. Any one of these linkers may be used in combination with or in place of another. In some cases, a linker can simply be a covalent bond (e.g., a disulfide bond) or a noncovalent bond (e.g., an ionic bond) between the second single-stranded nucleic acid and the solid support.

The following examples are provided for the purpose of illustration and not limitation.

### EXAMPLES

### Example 1

### USAT Assembly

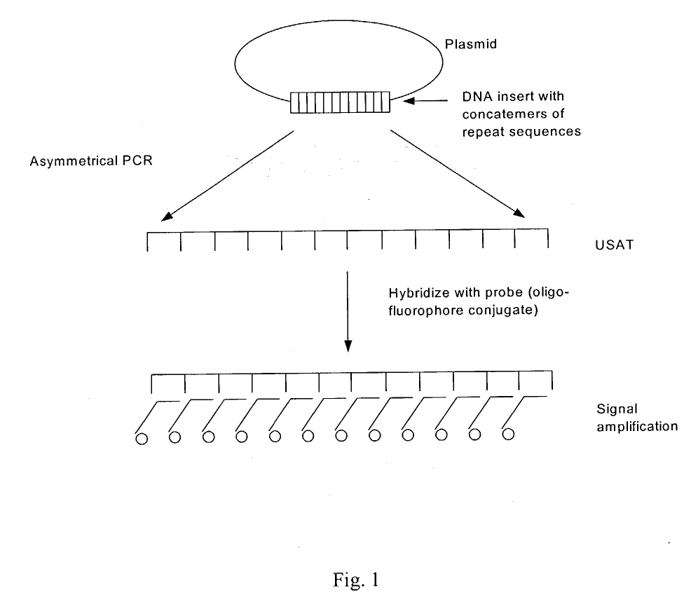

The USAT may be assembled by generating a single-stranded polynucleotide tail with a defined number of concatemer sequences by recombinant DNA technology, as shown in FIG. 1. A double stranded DNA fragment of repeat sequences can be generated and cloned into a plasmid vector for further amplification. The amplified and defined repeat sequence can then serve as a template for generating ssDNA through asymmetrical PCR. Each repeat sequence can then act as a binding site for the labeled probe.

### Example 2

### USAT Assembly

A naturally occurring repeat sequence (such as an Alu sequence or a Mini-satellite sequence) with non-repetitive flanking sequences, or any synthetically assembled sequences containing one or more types of desired repeat sequences may be used as a template for conventional PCR. After the first round of amplification, another round of one-directional or single primer extension can be used for generating the single-stranded DNA for attachment to an antibody or other defined nucleic acid sequence. FIG. 2 is an exemplary procedure of isolating repeating Alu sequence from human genome.

### Example 3

### Attachment of USAT to Antibody

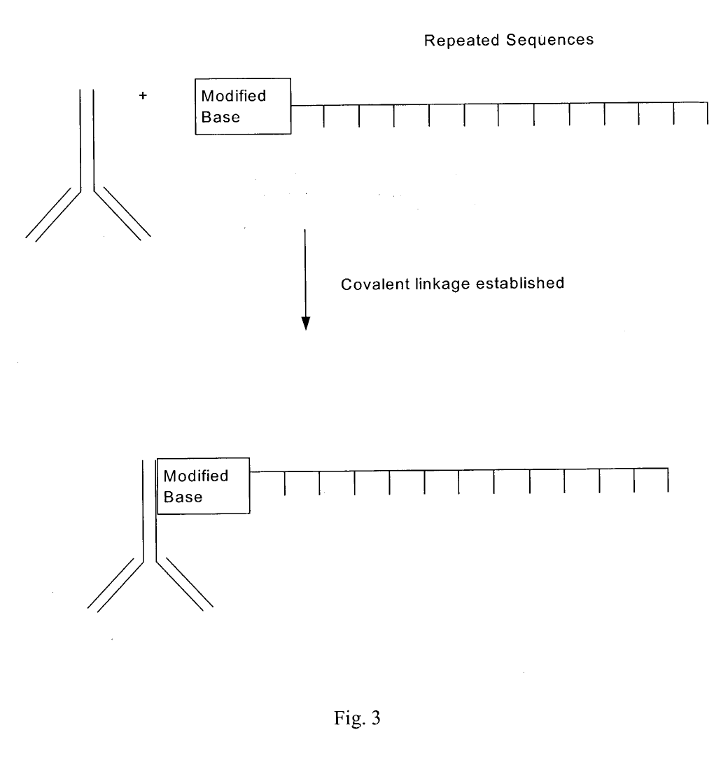

Modified base at end of USAT is used for covalent attachment with antibody. Multiple USAT's with a modified base may also be attached to the antibody, as illustrated in FIG. 3.

### Example 4

### Attachment of USAT to Antibody

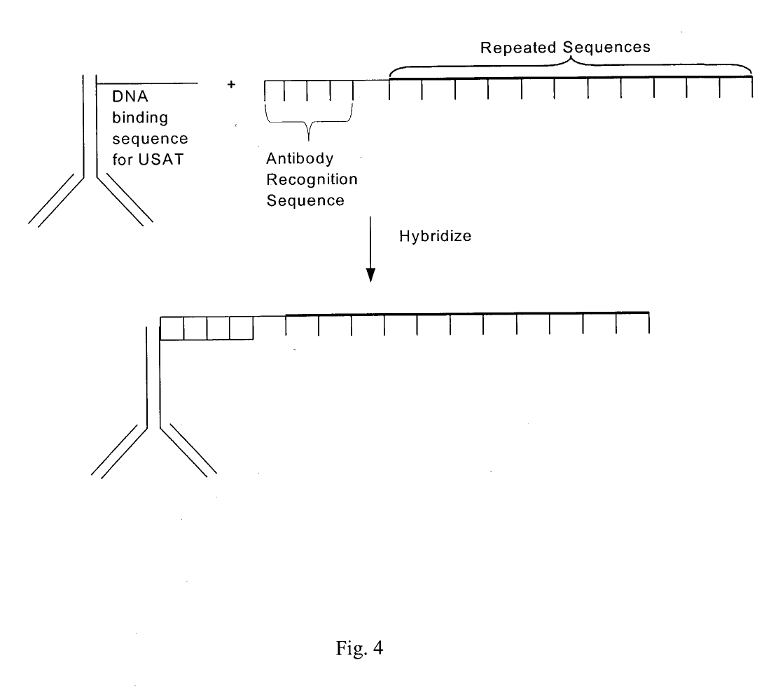

Hybridize USAT to antibody previously modified with defined sequence of DNA. Multiple DNA binding sequences may also be attached to the antibody as depicted in FIG. 4.

### Example 5

### Detection Signals Using Difference Probe Combinations in ELISA

A repetitive sequence from human genome was tested as the first single-stranded nucleic acid or USAT in a solid matrix format, where the repetitive sequence was attached to the surface of a 96-well plate. Different versions of second single-stranded nucleic acids (probes) of varying lengths and with different number of biotin moieties as detectable labels were generated. Hybridization of the probes to the USAT and signal amplification were monitored calorimetrically using a streptavidin-horseradish peroxidase (SA-HRP) conjugate in an enzyme-linked immunosorbent assay (ELISA) format.

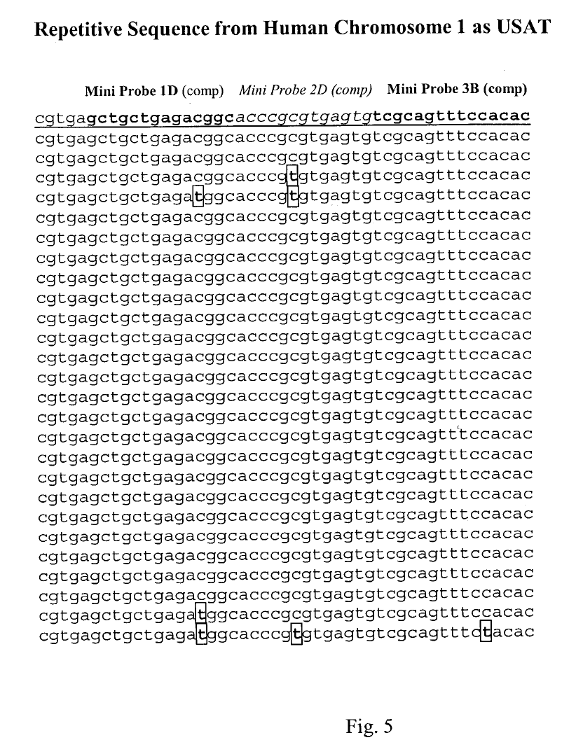

FIG. 5 shows a USAT sequence constructed from a representative sequence in human genome (Locus: HSY13542, Accession: Y13543, bases: 15809-17080) with a repeat sequence CEB92 of 47 bp (underlined). Polymorphisms in the repeat sequences are shown in bold typeface and in boxes.

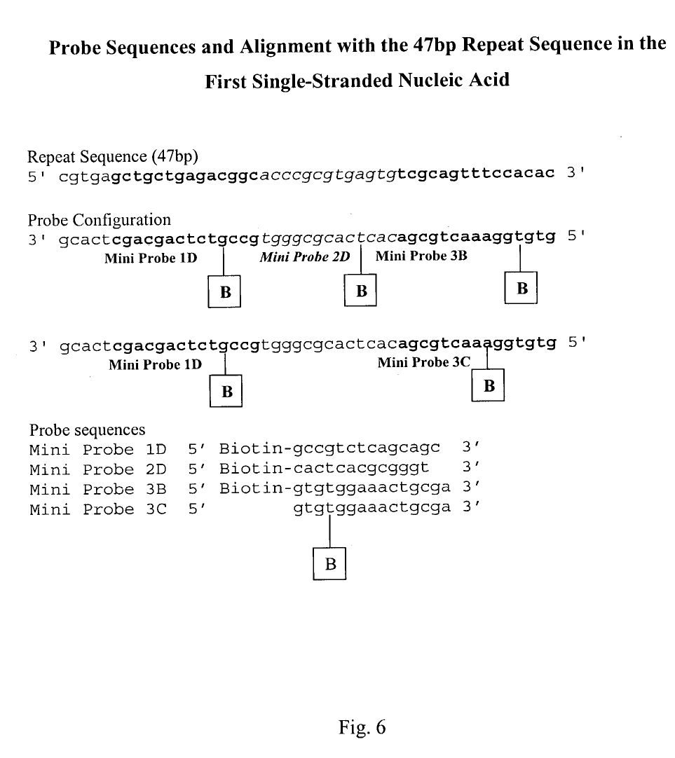

Probes, or the second single-stranded nucleic acids, are short (13-15 bases) single-stranded DNA probes labeled with biotin and shown in FIG. 6.

Results:

Single-stranded DNA sequence as USAT was reactive toward the various probes tested. Probe signal was not strictly additive and the various probes did not yield the same level of signal amplification. Increasing the number of probes within the 47 bp repeat sequence generally enhances the overall signal amplification.

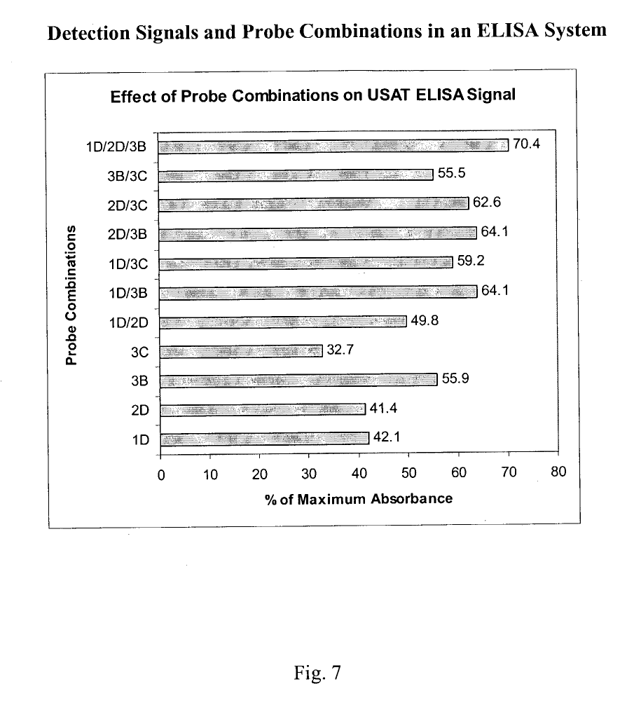

The values in FIG. 7 are average percentages of probe signals divided by the maximum signals obtained from each assay as determined by the highest values from the Assay Validity Control strips. Performance of the detection scheme is determined by positive and negative controls in the 8 well Assay Validity Control (AVC) strip. The AVC reagent contains a 40 bp synthetic DNA duplex that contains a biotinylated DNA strand. The AVC DNA is denatured and hybridized to the wells in the AVC strip. Hybridization is monitored by the well-established SA-HRP detection method. Two wells (A and B) in the control strip are for monitoring non-specific binding of the SA-HRP conjugate to the wells. Two wells (C and D) contain an immobilized oligonucleotide with a sequence that is not homologous to the AVC synthetic DNA. Two wells (E and F) contain an immobilized oligonucleotide that produces perfect base pairing when hybridized with the biotinylated DNA strand of the AVC. Two wells (G and H) contain an immobilized biotinylated oligonucleotide with a sequence that is not homologous to the AVC. The maximum signal is obtained from wells G and H. Slightly less signal is typically obtained form wells E and F. Wells A-D are negative controls with no signal production. The same quantity of USAT DNA was absorbed in all of the wells. All probes were pre-diluted to the same concentration and the equal volume of the probes were used for each assay.

### Example 6

### Cloning Strategy for Acquiring a First Single-Stranded Nucleic Acid

A recombinant DNA cloning process is used to acquire a 103 bp DNA sequence. A core 62 bp sequence within a DNA template is amplified by two primers containing restriction sites. A USAT sequence is generated through self-ligation of the core sequence, i.e., the repeat sequence, following an enzymatic digestion using appropriate restriction endonuclease(s).

Listed below are some exemplary primers and USAT sequences. The repeat sequence is underlined and restriction sites are in bold and Italic.

Template: USAT62 (Repeat Sequence)

5′ ACG GGG TCA GAC GCT CAA TGG TTC GAT CAC
ACA CGT TAA GGG ATT TTG GTC ATG AGA TTA TC 3′

Sense primer (Nco I restriction site in bold and Italic with “\hat{ }” indicating cleavage site) 5′ TAG TAA TCA AGT TC\hat{ }CATG GAC GGG GTC AGA CGC TCA 3′

Anti-sense primer (Nco I restriction site in bold and Italic with “\hat{ }” indicating cleavage site) 5′ TAG TAA TCA AGT TC\hat{ }C ATG GCT AGA TAA TCT CAT GAC CAA AAT CC 3′

Complete USAT sequence with a single repeat sequence (103 bases)

5′ TAG TAA TCA AGT TCC ATG GAC GGG GTC AGA CGC TCA
ATG GTT CGA TCA CAC ACG TTA AGG GAT TTT GGT CAT
GAG ATT ATC TAG CCA TGG AAC TTG ATT ACT A 3′

Other sequences with different restriction enzyme sites can be added to the 5′ end of the primers. An additional restriction site is added to the new primers for every cloning cycle. After each round of cloning, the region flanking the core sequence will become longer, but the repeat sequence will not change.

What is claimed is:

1. A detection agent, comprising a linear first single-stranded nucleic acid attached to a capture moiety that specifically binds to a target molecule, wherein said first single-stranded nucleic acid comprises a plurality of repeat sequences, each of which is a non-homopolymeric polynucleotide and specifically binds to a second single-stranded nucleic acid comprising a detectable label.

2. The detection agent of claim 1, wherein said capture moiety is a nucleic acid, a protein, or a carbohydrate.

3. The detection agent of claim 2, wherein said capture moiety is an antibody.

4. The detection agent of claim 1, wherein each of said repeat sequences is about 5 to about 100 bases in length.

5. The detection agent of claim 4, wherein each of said repeat sequences is about 50 bases in length.

6. The detection agent of claim 1, wherein said second single-stranded nucleic acid comprises multiple detectable labels.

7. The detection agent of claim 1, wherein said repeat sequences are contiguous.

8. The detection agent of claim 1, wherein said linear first single-stranded nucleic acid comprises SEQ ID NO:1, each of said repeat sequences is SEQ ID NO:2, said target molecule is Thyroid Stimulating Hormone (TSH), said capture moiety comprises an antibody against TSH, and said second single-stranded nucleic acid has three detectable labels.

9. A method for amplifying detection signals for a target molecule, comprising the steps of:
(a) contacting a detection agent with a sample, said detection agent comprising a linear first single-stranded nucleic acid attached to a capture moiety that specifically binds to a target molecule, wherein said first single-stranded nucleic acid comprises a plurality of repeat sequences, each of which is a non-homopolymeric polynucleotide and specifically binds to a second single-stranded nucleic acid comprising a detectable label; and
(b) contacting said detection agent with said second single-stranded nucleic acid under suitable conditions such that said repeat sequences specifically bind to said second single-stranded nucleic acid.

10. The method of claim 9, wherein said capture moiety is a nucleic acid, a protein, or a carbohydrate.

11. The method of claim 10, wherein said capture moiety is an antibody.

12. The method of claim 9, wherein each of said repeat sequences is about 5 to about 100 bases in length.

13. The method of claim 12, wherein each of said repeat sequences is about 50 bases in length.

14. The method of claim 9, wherein said second single-stranded nucleic acid comprises multiple detectable labels.

15. The method of claim 9, wherein said repeat sequences are contiguous.

16. The method of claim 9, wherein said linear first single-stranded nucleic acid comprises SEQ ID NO:1, each of said repeat sequences is SEQ ID NO:2, said target molecule is Thyroid Stimulating Hormone (TSH), said capture moiety comprises an antibody against TSH, and said second single-stranded nucleic acid has three detectable labels.

17. A kit for amplifying detection signals for a target molecule, comprising a detection agent that comprises a linear first single-stranded nucleic acid, which is attached to a capture moiety that specifically binds to the target molecule, and a second single-stranded nucleic acid comprising a detectable label, wherein said first single-stranded nucleic acid comprises a plurality of repeat sequences, each of which is a non-homopolymeric polynucleotide and specifically binds to the second single-stranded nucleic acid.

18. The kit of claim 17, wherein said capture moiety is a nucleic acid, a protein, or a carbohydrate.

19. The kit of claim 18, wherein said capture moiety is an antibody.

20. The kit of claim 17, wherein each of said repeat sequences is about 5 to about 100 bases in length.

21. The kit of claim 20, wherein each of said repeat sequences is about 50 bases in length.

22. The kit of claim 17, wherein said second single-stranded nucleic acid comprises multiple detectable labels.

23. The kit of claim 17, wherein said repeat sequences are contiguous.

24. The kit of claim 17, wherein said linear first single-stranded nucleic acid comprises SEQ ID NO:1, each of said repeat sequences is SEQ ID NO:2, said target molecule is Thyroid Stimulating Hormone (TSH), said capture moiety comprises an antibody against TSH, and said second single-stranded nucleic acid has three detectable labels.


SEQUENCE LISTING (13 sequences)

SEQ ID NO: 1 - DNA, 1269 bp, Homo sapiens
  repetitive sequence from human chromosome 1 as Universal Signal Amplification Tail (USAT)
cgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgtgtgagtgtcgcagtttccacaccgtgagctgctgagatggcacccgtgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagacggcacccgcgtgagtgtcgcagtttccacaccgtgagctgctgagatggcacccgcgtgagtgtcgcagtttccacaccgtgagctgctgagatggcacccgtgtgagtgtcgcagtttctacac

SEQ ID NO: 2 - DNA, 47 bp, Homo sapiens
  repeat_unit (1)..(47): 47bp repeat sequence from human chromosome 1
cgtgagctgctgagacggcacccgcgtgagtgtcgcagtttccacac

SEQ ID NO: 3 - DNA, 47 bp, Artificial Sequence
  Description of Artificial Sequenceprobe configuration
gtgnggaaactgcgacacncacgcgggtgccntctcagcagctcacg

SEQ ID NO: 4 - DNA, 47 bp, Artificial Sequence
  Description of Artificial Sequenceprobe configuration
gtgtggnaactgcgacactcacgcgggtgccntctcagcagctcacg

SEQ ID NO: 5 - DNA, 14 bp, Artificial Sequence
  Description of Artificial SequenceMini Probe 1D
nccgtctcagcagc

SEQ ID NO: 6 - DNA, 13 bp, Artificial Sequence
  Description of Artificial SequenceMini Probe 2D
nactcacgcgggt

SEQ ID NO: 7 - DNA, 15 bp, Artificial Sequence
  Description of Artificial SequenceMini Probe 3B
ntgtggaaactgcga

SEQ ID NO: 8 - DNA, 15 bp, Artificial Sequence
  Description of Artificial SequenceMini Probe 3C
gtgnggaaactgcga

SEQ ID NO: 9 - DNA, 21 bp, Artificial Sequence
  Description of Artificial Sequencenon-homopolymeric repeat sequence
atgcgatatgcgatatgcgat

SEQ ID NO: 10 - DNA, 62 bp, Artificial Sequence
  Description of Artificial Sequencetemplate USAT62 repeat sequence
acggggtcagacgctcaatggttcgatcacacacgttaagggattttggtcatgagattatc

SEQ ID NO: 11 - DNA, 36 bp, Artificial Sequence
  Description of Artificial Sequencesense primer
tagtaatcaagttccatggacggggtcagacgctca

SEQ ID NO: 12 - DNA, 44 bp, Artificial Sequence
  Description of Artificial Sequenceanti-sense primer
tagtaatcaagttccatggctagataatctcatgaccaaaatcc

SEQ ID NO: 13 - DNA, 103 bp, Artificial Sequence
  Description of Artificial Sequencecomplete USAT sequence with a single repeat sequence
tagtaatcaagttccatggacggggtcagacgctcaatggttcgatcacacacgttaagggattttggtcatgagattatctagccatggaacttgattacta